(19) United States
(12) Patent Application Publication
(10) Pub. No.: US 2008/0139893 A1
(43) Pub. Date: Jun. 12, 2008

(54) Apparatus And System For Sensing and Analyzing Body Sounds

(76) Inventors: Warren Lee, Niskayuna, NY (US); Weston Blaine Griffin, Guilderland, NY (US); Mirsaid Seyed Bolorforosh, Guilderland, NY (US); Kai Erik Thomenius, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/608,294

(22) Filed: Dec. 8, 2006

(51) Int. Cl. A61B 5/00

(52) U.S. Cl. 600/300

(57) ABSTRACT

An apparatus and system for sensing body sounds of a subject is described. The apparatus includes a flexible sheet for conformally contacting at least a portion of a subject's body surface proximate a body region generating body sounds. The apparatus also includes a plurality of acoustic sensors formed in the sheet for sensing body sounds from the subject and generating respective signals indicative of the sensed body sounds.

### FIELD OF THE INVENTION

The present disclosure relates generally to medical diagnostic devices. More particularly, the present disclosure relates to an apparatus and system for sensing and analyzing body sounds.

### BACKGROUND OF THE INVENTION

Pulmonary imaging devices that sense vibrational energy generated by a subject's lungs have been used to generate functional images of the lungs. Conventional systems use a limited number of discrete microphones or microphone arrays applied to the subject's body surface proximate the lungs to sense the vibrational energy, typically in the form of body sounds. The sounds are further processed to generate two-dimensional (2D) images that may be used for disease diagnosis.

Piezoelectric materials, such as polyvinylidene (PVDF), have been used in electronic stethoscopes to sense acoustic signals corresponding to a strain induced in the material responsive to the acoustic signal. The induced strain generates an electrical signal proportional to a strength of the acoustic signal causing the strain. Typically, such piezoelectric materials are not piezoelectrically active until random ferroelectric domains of the material are aligned. Alignment of the ferroelectric domains may be accomplished using the known technique of poling. Poling typically includes inducing a direct current voltage across the material to align the ferroelectric domains according to an induced electric field, resulting in a net piezoelectric effect. Poling may be controlled by varying a voltage, a temperature, and/or a time of voltage application to achieve a desired piezoelectric effect.

### BRIEF DESCRIPTION OF THE INVENTION

In an example embodiment, the invention includes an apparatus for sensing body sounds of a subject. The apparatus includes a flexible sheet for conformally contacting at least a portion of a subject's body surface proximate a body region generating body sounds. The apparatus also includes a plurality of acoustic sensors formed in the sheet for sensing body sounds from the subject and generating respective signals indicative of the sensed body sounds.

In another example embodiment, the invention includes a system for sensing body sounds of a subject. The system includes a flexible sheet for conformally contacting at least a portion of a subject's body and an array of acoustic sensors formed within the sheet for sensing body sounds from the subject and generating respective signals indicative of the sensed body sounds. The system also includes an electrode network formed within the sheet in communication with respective sensors for conducting the respective signals away from the sheet and a processor receiving the respective signals for processing the signals to generate an image of the body sounds.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 shows an example embodiment of an apparatus and system for sensing body sounds of a subject;

FIG. 2 shows an example embodiment of the apparatus positioned on a subject for body sound sensing;

FIG. 3 shows a cross-sectional view of an example embodiment of the apparatus taken along lines 3-3 of FIG. 1;

FIG. 4 shows another example embodiment of an apparatus for sensing body sounds of a subject;

FIG. 5 shows another example embodiment of an apparatus for sensing body sounds of a subject configured as a garment; and

FIG. 6 shows how an example embodiment of the apparatus of FIG. 1 receives sound from a body region; and

FIG. 7 shows a schematic diagram of an example processor of the system of FIG. 1.

### DETAILED DESCRIPTION OF THE INVENTION

Previous body sound diagnostic systems, such as electronic stethoscopes or lung imaging systems, have typically relied on discrete sensors or a limited number of sensors to sense body sounds. Consequently, these systems are not readily adapted for performing signal processing techniques that are commonly used in other sensing technologies, such as ultrasound sensing. For example, signal processing techniques based on time of arrival differences of a signal may be used to generate three dimensional (3D) information about a source of the signal. However, such techniques typically require a relatively large number, i.e. a relatively high density, of sensing elements in a sensing array to support advanced signal processing.

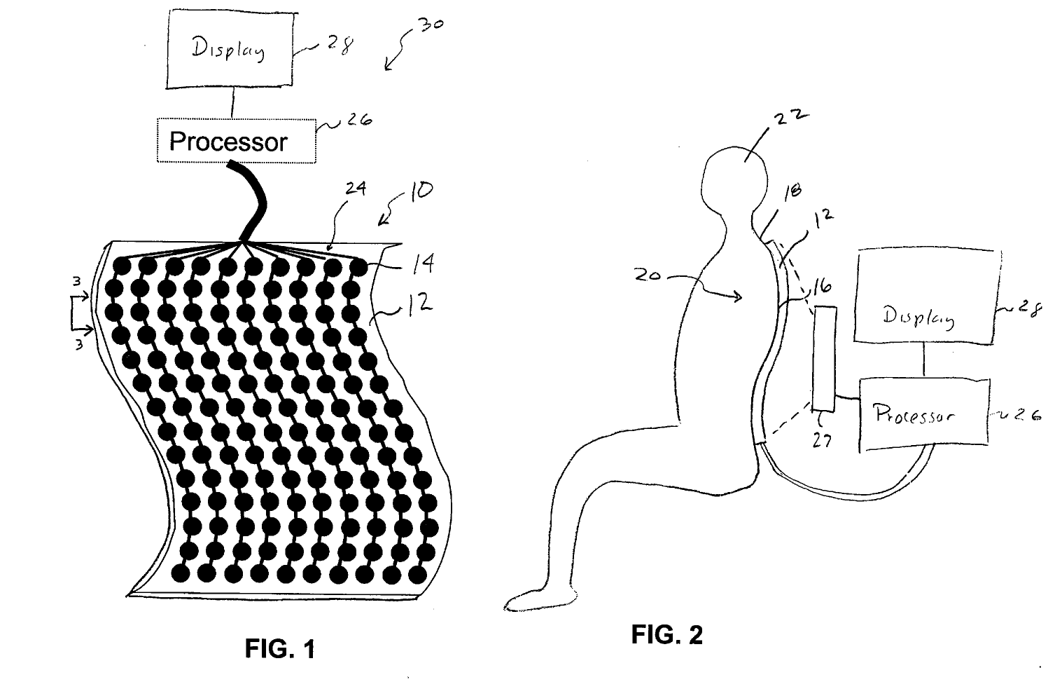

To provide improved body sound sensing, the inventors have developed a flexible sheet of acoustic sensors for conformally contacting a subject's body surface for sensing body sounds. FIG. 1 illustrates an example embodiment of an apparatus 10 for sensing body sounds of a subject. The apparatus includes a flexible sheet 12 and a plurality, such as an array, of acoustic sensors 14 formed in the sheet 12 for sensing body sounds from the subject and generating respective signals indicative of the sensed body sounds. As shown in FIG. 2, the flexible sheet 12 may be configured for conformally contacting at least a portion 16, such as a back portion, of a subject's body surface 18 proximate a body region 20 generating body sounds, such as a pulmonary region of the subject 22. Returning to FIG. 1, sheet 12 may include an electrode network 24 formed within the sheet 12 in communication with respective sensors 14 for conducting the respective signals away from the sheet 12. The apparatus 10 may be connected to a processor 26 receiving the respective signals via the network 24 for processing the signals to generate an image on display 28, thereby forming an acoustic imaging system 30. It is envisioned that such an apparatus may be effective for sensing many different types of body sounds, such as breathing sounds from a subject's lungs, blood vessel sounds (e.g., bruits), cardiac sounds from a subject's heart, and/or digestive sounds.

In an example embodiment, the sheet 12 may include a piezoelectric material, such as a polyvinylidene (PVDF) material or a polyvinylidene-triflouroethylene (PVDF-TrFE) material. The sensors 14 may include activated regions within the piezoelectric material. The activated regions may be configured using known poling techniques so that a strain induced at the poled regions by an acoustic pressure, for example, body sounds, generates an electrical signal proportional to a strength of the sounds causing the strain. The electrical signal generated by the activated regions may be conducted to the processor 26 via network for processing to create an image of the sensed sounds. Such activated regions may be used to form a relatively dense acoustic sensor array the may allow advanced signal processing techniques to be performed on data gathered from the sensors 14 formed by the respective activated regions.

In another example embodiment, the acoustic sensors 14 may be formed from piezoelectric elements such as a polyvinylidene (PVDF) material or a polyvinylidene-triflouroethylene (PVDF-TrFE) material. The sensors 14 may be formed on or within the sheet 12. FIG. 3 shows a cross sectional view of an example embodiment of the sheet of FIG. 1 taken along line 3-3. As shown in FIG. 3, the sheet 12 may include a first layer 34 and a second layer 32, wherein the acoustic sensor 14 is disposed between the first layer 34 and the second layer 32. The first layer 34 may include an aperture 36 formed therethrough for exposing a top side 38 of the acoustic sensor 14. The second layer 32 may include a curved region 40 underneath a bottom side 42 of the acoustic sensor 14 so that the acoustic sensor 14 is supported in a curved configuration. As shown in FIG. 3, the curved region 40 may be convex with respect to aperture 36 so that the top side 38 is curved towards the aperture 36. In another embodiment, the curved region 40 may be concave with respect to aperture so that the top side 38 is curved away from the aperture 36. For readers desirous of background information regarding this example embodiment, reference is made to a paper entitled “Contact-type Vibration Sensors using Curved Clamped PVDF Film”, published in IEEE Sensors Journal, Vol. 6, No. 5, October 2006, which paper is incorporated herein by reference in its entirety. It should be appreciated that one skilled in the art may use other types of acoustic sensors to sense body sounds, such as micro-electromechanical (MEM) sensors that may include capacitive micro-machined transducers.

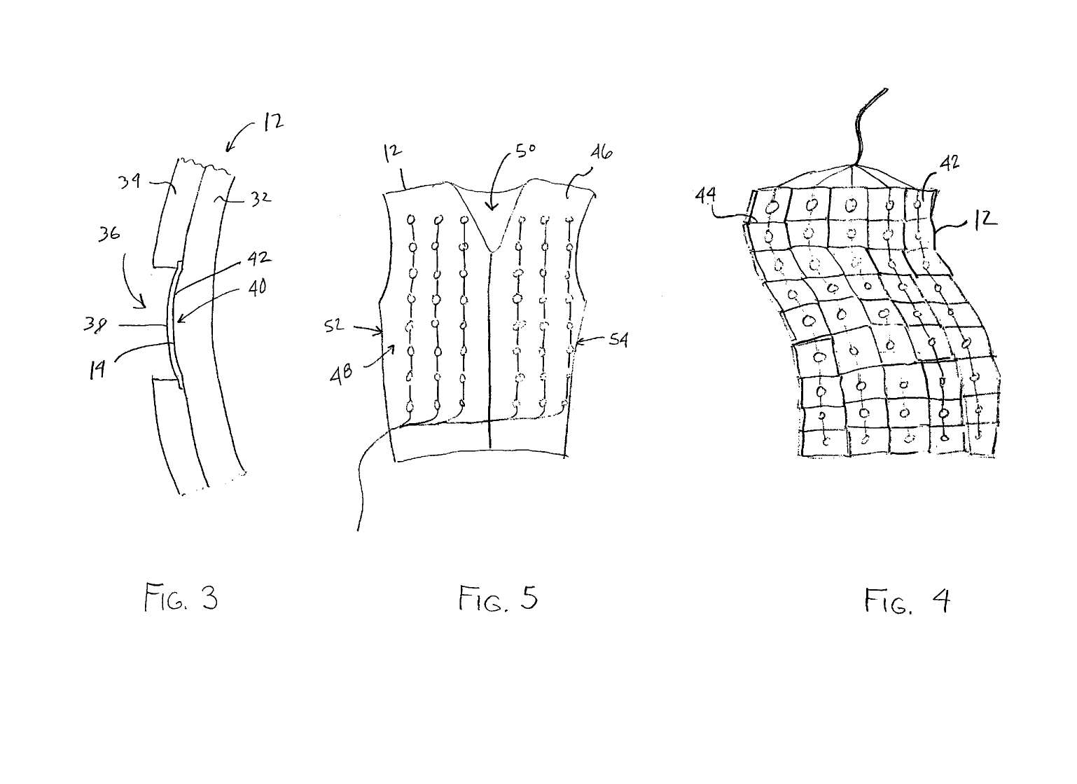

In an aspect of the invention shown in FIG. 4, the flexible sheet may include a plurality of movably connected, relatively rigid segments 42. Although the individual segments may be relatively rigid, the sheet, owing to movable connections 44, such as a flexible material connected between adjacent segments, may be configured to conform to a body surface 18. Each of the rigid segments 42 may include at least one acoustic sensor 14, such as one or more of the piezoelectric sensors embodiments described previously. In another aspect of the invention depicted in FIG. 5, the sheet 12 may include a garment wearable by a subject. The garment may be shaped to cover a body surface portion 16 proximate a body region 20 generating body sounds. For example, the garment may include a vest for placing around a subject's thoracic region. The vest may include a plurality of acoustic sensors 14, such as one or more of the piezoelectric sensors embodiments described previously, formed in or on the vest. The sensors may be arranged in the front 48, back 50, and/or respective sides 52, 54 of the vest.

If the body sounds sensed by the system include higher order harmonics, such as harmonics greater than 10 kilohertz (kHz), it is envisioned that the body sound sensing apparatus 10 as described above that includes a sufficiently dense sensor array may be used to provide 3D imaging, for example, using known signal processing techniques. In addition, a three dimensional position of the sensors 14 of FIG. 1 would need to be determined to allow beamforming. For example, a prior art optical tracking method employing an optical tracker 27 as shown in FIG. 2 may be used to provide position information of the sensors 14 to the processor 26. As shown in FIG. 1, the system 30 may include the flexible sheet 12 for conformally contacting at least a portion 16 of a subject's body and an array of acoustic sensors 14 formed within the sheet. The array of acoustic sensors 14 may be sufficiently dense so as to provide a resolution for allowing three dimensional imaging of received sounds, such as harmonics of body sounds greater than about 10 kHz. The system 30 may also include an electrode network 24 formed within the sheet 12 in communication with respective sensors 14 for conducting the respective signals away from the sheet 12. A processor 26 may receive the respective signals and be configured for processing the signals to generate a three dimension image of body sounds.

Three dimensional images may be generated using known processing techniques based on arrival time differences from a point source P within a body region. As shown in FIG. 6, sound signals from the point P are propagated along path P₁ . . . Pₙ to respective sensors 14₁ . . . 14ₙ. Because the path lengths may differ depending on distance of the point from the respective sensors, sounds signals from the point P will arrive at different times at the respective sensors. This phenomenon may allow processing of the signals to extract 3D information when harmonics of the signals exceed about 10 kHz.

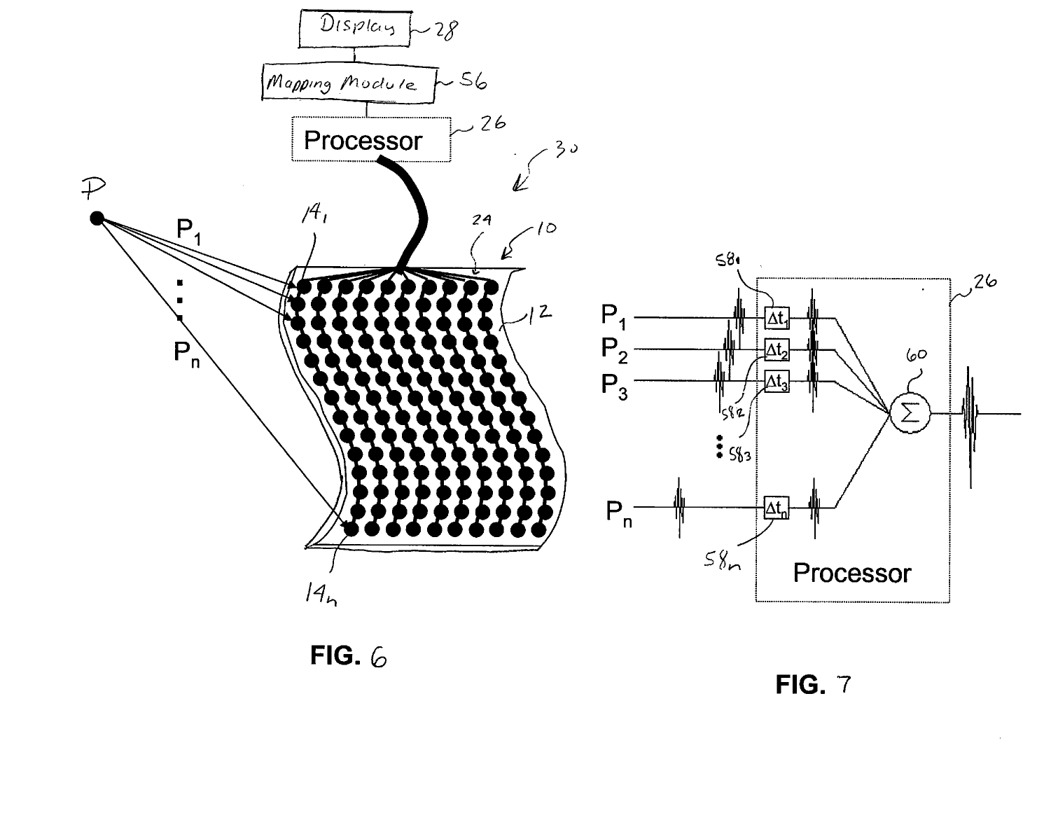

In an embodiment depicted in FIG. 7, the processor 26 may include delay element modules 58₁ . . . 58ₙ for adjusting arrival times of the respective signals from a point P with respect to one another. The processor 26 may also include a summing module 60 for adding at least some of the signals together after their arrival times have been adjusted. The resulting signal may be further processed to increase a signal to noise ratio (SNR) and/or generate 3D information using techniques known in the art.

In another embodiment, the system 30 may include a mapping module 56 for enhancing an image being generated by the system. The mapping module 58 may be configured to receive processed image information from the processor 26, such as image values for respective pixels of an image, and map these values using a mapping function to generate different values for the pixels that provide an improved image, such as an enhanced contrast image. In an embodiment of the invention, the mapping module 58 may be configured for providing non-linear mapping, such as by using a gamma curve.

While certain embodiments of the present invention have been shown and described herein, such embodiments are provided by way of example only. Numerous variations, changes and substitutions will occur to those of skill in the art without departing from the invention herein. Accordingly, it is intended that the invention be limited only by the spirit and scope of the appended claims.

What is claimed is:

1. An apparatus for sensing body sounds of a subject comprising:
a flexible sheet for conformally contacting at least a portion of a subject's body surface proximate a body region generating body sounds; and
a plurality of acoustic sensors formed in the sheet for sensing body sounds from the subject and generating respective signals indicative of the sensed body sounds.

2. The apparatus of claim 1, wherein the flexible sheet comprises a piezoelectric material.

3. The apparatus of claim 2, wherein the sensors comprise activated regions within the piezoelectric material.

4. The apparatus of claim 1, further comprising an electrode network formed within the sheet in communication with the sensors for conducting the respective signals away from the sheet.

5. The apparatus of claim 1, wherein the acoustic sensors are formed from piezoelectric elements.

6. The apparatus of claim 5, wherein the piezoelectric elements are fabricated from a polyvinylidene (PVDF) material.

7. The apparatus of claim 5, wherein the piezoelectric elements are fabricated from a polyvinylidene-triflouroethylene (PVDF-TrFE) material.

8. The apparatus of claim 1, wherein the sheet comprises a first and second layer.

9. The apparatus of claims 8, wherein the acoustic sensors are disposed between the first and second layer.

10. The apparatus of claim 9, wherein the first layer comprises apertures exposing respective top sides of the acoustic sensors.

11. The apparatus of claim 10, wherein the second layer comprises curved regions underneath respective bottom sides of the acoustic sensors so that the acoustic sensors are supported in a curved configuration.

12. The apparatus of claim 11, wherein the curved regions are convex with respect to the apertures.

13. The apparatus of claim 11, wherein the curved regions are concave with respect to the apertures.

14. The apparatus of claim 1, wherein the flexible sheet comprises a plurality of movably connected relatively rigid segments.

15. The apparatus of claim 1, wherein each of the rigid segments comprises at least one acoustic sensor.

16. The apparatus of claim 1, wherein the sheet comprises a garment wearable by the subject.

17. The apparatus of claim 16, wherein the garment comprises a vest.

18. The apparatus of claim 1, wherein the acoustic sensors comprise micro-electromechanical sensors.

19. The apparatus of claim 18, wherein the micro-electromechanical sensors comprise capacitive micro-machined transducers.

20. An apparatus for sensing body sounds of a subject comprising:
a flexible sheet comprising a polyvinylidene (PVDF) material for conformally contacting at least a portion of a subject's body surface proximate a body region generating body sounds; and
an array of activated regions formed in the sheet for sensing body sounds from the subject and generating respective signals indicative of the sensed body sounds.

21. The apparatus of claim 20, wherein the array of acoustic sensors is sufficiently dense to provide a resolution for allowing three dimensional imaging of the sounds.

22. A system for sensing body sounds of a subject comprising:
a flexible sheet for conformally contacting at least a portion of a subject's body;
an array of acoustic sensors formed within the sheet for sensing body sounds from the subject and generating respective signals indicative of the sensed body sounds
an electrode network formed within the sheet in communication with respective sensors for conducting the respective signals away from the sheet; and
a processor receiving the respective signals for processing the signals to generate an image of the body sounds.

23. The system of claim 22, wherein the processor further comprises a mapping module for enhancing the image.

24. The system of claim 23, wherein the mapping module is configured for performing non-linear mapping.

25. The system of claim 24, wherein the non-linear mapping comprises use of a gamma curve.

26. The system of claim 22, wherein the array of acoustic sensors is sufficiently dense to provide a resolution for generating a three dimensional image of the sounds.

27. The system of claim 26, wherein the processor comprises delay element modules for adjusting arrival times of the respective signals with respect to one another.

28. The system of claim 27, wherein the processor further comprises a summing module for adding at least some of the signals together after their arrival times have been adjusted.

29. The system of claim 22, further comprising an optical tracker in communication with the processor for determining respective three dimensional positions of the sensors in the array.
